(19) United States
(12) Patent Application Publication
(10) Pub. No.: US 2005/0047846 A1
(43) Pub. Date: Mar. 3, 2005

(54) Single-use applicators for adhesive material, packaging systems, methods of use and methods of manufacture

(75) Inventors: Upvan Narang, Raleigh, NC (US); Gabriel N. Szabo, Raleigh, NC (US); Jack Goodman, Ann Arbor, MI (US)

Correspondence Address: OLIFF & BERRIDGE, PLC, P.O. BOX 19928, ALEXANDRIA, VA, 22320, US

(73) Assignee: CLOSURE MEDICAL CORPORATION, Raleigh, NC (US)

(21) Appl. No.: 10/649,879

(22) Filed: Aug. 28, 2003

(51) Int. Cl. B43K 5/14; B43K 5/00; A61M 35/00

(52) U.S. Cl. 401/133; 401/205; 604/3

(57) ABSTRACT

A single use applicator for applying an adhesive material, particularly for medical use, includes a semi-rigid base strip having a longitudinal direction, and a reservoir containing an adhesive material, the reservoir being secured to one side of the base strip. The base strip is arranged to be folded about an axis substantially perpendicular to the longitudinal direction such that a portion of the reservoir is ruptured and pressure is applied on the reservoir to expel the adhesive material from the ruptured reservoir. In embodiments, the base strip has a hole formed therethrough and a projection corresponding to the hole formed on one side thereof. The reservoir is disposed over the hole on a side of the base strip opposite the one side on which the projection is formed. When the base strip is folded, the projection extends into the hole to apply pressure on the reservoir.

### BACKGROUND OF THE INVENTION

This invention relates to single use applicators for applying an adhesive material, such as a polymerizable monomer compound, particularly for medical use. The present invention also relates to packaging systems and methods of use for such applicators.

Numerous swabs, applicators, dispensers and kits for dispensing and applying various materials, including adhesive materials, are known. However, these known arrangements possess various shortcomings that make them undesirable in many applications.

For example, U.S. Pat. No. 4,140,409 to DeVries discloses a disposable liquid applicator. A prescored container holds the liquid to be dispensed. When the container is bent away from the prescored portion and about itself, the liquid is dispensed into an absorbent material fastened to the container overlying the prescored portion.

U.S. Pat. No. 4,430,013 to Kaufman discloses a disposable swab article that comprises an applicator package. The applicator package has a foam applicator pad and an adjacent backing member. The backing member is a relatively rigid flat sheet material and has at least on reservoir. When the backing member is folded, a slit or weakened portion formed in the backing member is ruptured and the contents of the reservoir is released by pressure caused by deformation of the backing member.

U.S. Pat. No. 4,519,795 to Hitchcock, Jr. et al. discloses a disposable swab for application of a medicament to the skin. The swab comprises a flat, sheet-like strip and a pad member centrally attached to at least one side of the strip. Fold lines on the strip allow opposed terminal portions of the strip to be folded rearwardly relative to the pad member and allow opposite ends of the strip to be pinched together into flat, abutting relation. The strip thus forms a generally triangular structure with the pad member carried on the base of the triangular structure. Aluminum foil may be laminated over the pad member on the strip.

U.S. Pat. No. 4,812,067 to Brown et al. discloses a disposable applicator package for application of flowable materials to surfaces. The applicator package comprises a backing member comprising a first flexible sheet, an applicator pad on the backing member, a second member in contact with the backing member and comprising a formed semi-rigid sheet having at least one recess adjacent the pad, at least one cavity or reservoir distally spaced from the recess, a peripheral groove in fluid connection with the recess, co-planar separations between the cavity, the groove and the recess, and an opening between the pad and the recess. Pressure applied to a bottom wall of the cavity to break seals between the cavity and the groove and the recess so that fluid in the cavity flow to the recess. The opening between the recess and the pad is opened by bending the backing member and applying pressure to the recess.

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and polymers, they have found wide use due to the speed with which they cure, the strength of the resulting bond formed, and their relative ease of use. These characteristics have made the α-cyanoacrylate adhesives the primary choice for numerous applications such as bonding plastics, rubbers, glass, metals, wood, and, more recently, biological tissues.

It is known that monomeric forms of α-cyanoacrylates are extremely reactive, polymerizing rapidly in the presence of even minute amounts of an initiator, including moisture present in the air or on moist surfaces such as animal (including human) tissue. Monomers of α-cyanoacrylates are anionically polymerizable or free radical polymerizable, or polymerizable by zwitterions or ion pairs to form polymers. Once polymerization has been initiated, the cure rate can be very rapid.

Medical applications of 1,1-disubstituted ethylene adhesive compositions include use as an alternate or an adjunct to surgical sutures and staples in wound closure as well as for covering and protecting tissue wounds such as lacerations, abrasions, burns, stomatitis, sores, and other open surface wounds. When such an adhesive is applied, it is usually applied in its monomeric form, and the resultant polymerization gives rise to the desired adhesive bond.

However, due to the need to apply the adhesive in its monomeric form, and due to the rapid polymerization rate of the monomers, it has been very difficult to design effective and commercially viable applicators and packaging systems. Such packaging systems must counterbalance the competing requirements that the monomer not prematurely polymerize, that the monomer be easily applied, that the monomer polymerize at a desired rate upon application, and that the sanitary and/or sterile properties of the monomer and applicator be maintained. This requirement, that the sanitary and sterile condition of the monomer and applicator be maintained, is particularly important in medical applications, where the user and/or the patient desires a clean product so as not to introduce further bacteria or foreign matter into a wound site.

In order to meet the above requirements, various packaging systems for monomeric adhesives have been proposed. These systems include large bottles with a single applicator, such as a large single- or multi-use brush; small applicators such as small ampoules containing monomer, for example within an internal frangible vial, that can be expelled through an integral applicator; and the like. However, a problem with many of these applicator systems is that the product contains more adhesive material than is necessary for a particular use. Because of the rapid polymerization rate of the monomers, any unused adhesive must often be discarded, because the remaining monomer undergoes polymerization, often within the applicator, to render the applicator unusable.

A further problem in addressing the above requirements of adhesive applicators and packaging is the need to provide a stable monomer product. Due to their reactivity, cyanoacrylate monomers are prone to premature polymerization, which would render the product useless. Thus, industrial production of monomeric adhesive compositions has had to balance rapid cure rates and high bond strengths with shelf-life. The shelf-life of these adhesives is primarily related to stability (i.e., constancy of compositional nature), uncured physical properties, rate of cure of the adhesive, as well as final cured properties of the composition. For example, the shelf-life of a monomeric α-cyanoacrylate composition may be measured as a function of the amount of time the composition can be stored before unacceptable levels of polymerization, such as measured by viscosity increase, occur. Unacceptable levels are indicated by a level of polymerization product that reduces the usefulness of the composition in the application for which it is produced.

One proposed solution to this reduced shelf-life problem is to incorporate one or more stabilizers into the adhesive composition. For example, as disclosed in U.S. Pat. Nos. 3,559,652 to Banitt et al. and 5,582,834 to Leung et al., suitable stabilizers for medically useful α-cyanoacrylate compositions include Lewis acids such as sulfur dioxide, nitric oxide, and boron trifluoride, as well as free-radical stabilizers including hydroquinone, monomethyl ether hydroquinone, nitrohydroquinone, catechol, and monoethyl ether hydroquinone. The combination of the two anionic stabilizers sulfur dioxide and sulfonic acid is also known and is disclosed in, for example, British Patent Application GB 2 107 328 A.

However, while the proposed solution of adding stabilizers provides compositions that are more stable, a different problem arises. That new problem is that as the concentration of the added stabilizers increases in the composition, the cure rate of the composition tends to decrease. Thus, further components must be provided, such as in a separate composition, to be mixed with the adhesive composition (either directly or at the application site) to increase the polymerization rate of the monomer. Such additional materials, such as polymerization initiators or rate modifiers, increase the cost of the final composition, and may increase the complexity of use of the composition.

Known devices fail to provide a disposable applicator and package assembly that is optimized for convenient dispensing and application of adhesive materials on a variety of surfaces and structures. The known applicators are generally either optimized for delivery of other compositions or are inconvenient for use in conjunction with adhesives. Furthermore, such conventional devices and packaging generally do not address the competing needs of ease of use and adhesive stability prior to application.

### SUMMARY OF THE INVENTION

The present invention addresses the above needs by providing applicators and packaging systems that permit economical and efficient use of adhesive compositions. In embodiments of this invention, applicators and packaging systems are provided whereby single-use applicators are provided with single-use amounts of adhesive material, thereby avoiding waste of excess adhesive material. The applicators can be provided in multiple different sizes, to account for situations where more or less adhesive material may be required.

In embodiments, the adhesive composition is a polymerizable monomer and is packaged in a material that is compatible with the adhesive composition.

In other embodiments, the applicators include a polymerization initiator or accelerator for the adhesive material. The polymerization initiator or accelerator may be disposed in or on a tip or other part of the applicator. The tip or other portion of the applicator may be at least one of porous, absorbent and adsorbent in nature and the polymerization initiator or accelerator may be absorbed or adsorbed into the portion of the applicator. The applicators may also include a partition separating first and second compartments, for example, to keep the polymerization initiator or accelerator separated from the adhesive material prior to use. In embodiments, the partition may be a frangible barrier.

In further embodiments, the applicators contain the adhesive material in the first compartment and include a medicament or other additive in the second compartment.

In particular, various embodiments of this invention are directed to an applicator for applying an adhesive material, comprising: a semi-rigid base strip having a longitudinal direction, a hole formed through the base strip and a projection corresponding to the hole formed on one side of the base strip; and a reservoir containing an adhesive material, the reservoir being disposed over the hole on a side of the base strip opposite the one side on which the projection is formed; wherein the base strip is arranged to be folded about an axis substantially perpendicular to the longitudinal direction such that a portion of the reservoir is ruptured and the projection extends into the hole to apply pressure on the reservoir and expel the adhesive material from the ruptured reservoir.

In embodiments, the reservoir is adhered to the base strip at least on opposite sides of the portion of the reservoir that is to be ruptured. In such embodiments, the reservoir is ruptured under stress that is generated by stretching the reservoir as the base strip is folded.

In embodiments, an applicator tip is formed over at least the portion of the reservoir that is ruptured. The applicator tip may be one of a foam pad, a rolling ball, a brush, a mesh, a spatula, a sponge and a swab. In further embodiments, the applicator tip comprises a quenched foam.

In embodiments, the reservoir comprises a sachet. In embodiments, the sachet has an ultrasonic weld seal. In other embodiments, the sachet is sealed by an adhesive or is heat-sealed. Further, the sachet may be made of a material that is compatible with the adhesive material. For example, as discussed above, the material may provide acceptable stability and shelf-life to the adhesive composition without the need to add separate stabilizer materials when the adhesive material is a polymerizable monomer. In embodiments, the sachet is made of aluminum. In other embodiments, the sachet is made of a plastic, such as a halogenated polymer.

In embodiments, the sachet has a weakened portion that defines the portion of the reservoir that is ruptured. The weakened portion may comprise a slit formed in at least one edge of the sachet. Further, the weakened portion may comprise a pre-scoring.

In embodiments, the sachet has a formed portion that fits in the hole of the base strip. The formed portion of the sachet may or may not be secured to the base strip.

In embodiments, the applicator is provided with a packaging system. In particular, various embodiments provide a sealing film over at least the applicator tip of the applicator. In embodiments, the sealing film comprises a peel-off barrier.

Alternatively or additionally, various embodiments provide a sealed pouch that encloses the entire applicator. In embodiments, the sealed pouch comprises one of polyester (such as Mylar®), foil and paper. In embodiments, the sealed pouch is a bacterial barrier.

Various other features and advantages of this invention will become apparent upon review of the following detailed description of exemplary embodiments.

### BRIEF DESCRIPTION OF THE DRAWINGS

Exemplary embodiments of this present invention are described in detail below, with reference to the attached drawing figures, in which:

FIG. 1 is a perspective view of a first embodiment of this invention;

FIGS. 2 and 3 are exploded views of the embodiment of FIG. 1;

FIG. 4 is a top view of the embodiment of FIG. 1;

FIG. 5 is a side view of the embodiment of FIG. 1;

FIG. 6 is a bottom view of a the embodiment of FIG. 1;

FIG. 7 is a bottom view of a sachet according to the exemplary embodiment of FIG. 1;

FIG. 8 is an exploded view of the sachet of FIG. 7;

FIG. 9 is an exploded view of another exemplary embodiment of the sachet of FIG. 7;

FIG. 10 is a perspective view of a second embodiment of this invention;

FIG. 11 shows the embodiment of FIG. 1 in use;

FIG. 12 shows the embodiment of FIG. 9 in use; and

FIG. 13 illustrates an embodiment of a packaging system according to this invention.

### DETAILED DESCRIPTION OF EXEMPLARY EMBODIMENTS

In embodiments of this invention, an applicator, packaging system or kit is provided that includes one or more applicators or dispensers packaged with a corresponding quantity of polymerizable adhesive material. The applicator or packaging is sealed to prevent external contaminants from entering, at least in the area where the adhesive material is contained, and for medical applications, preferably the applicator tip as well.

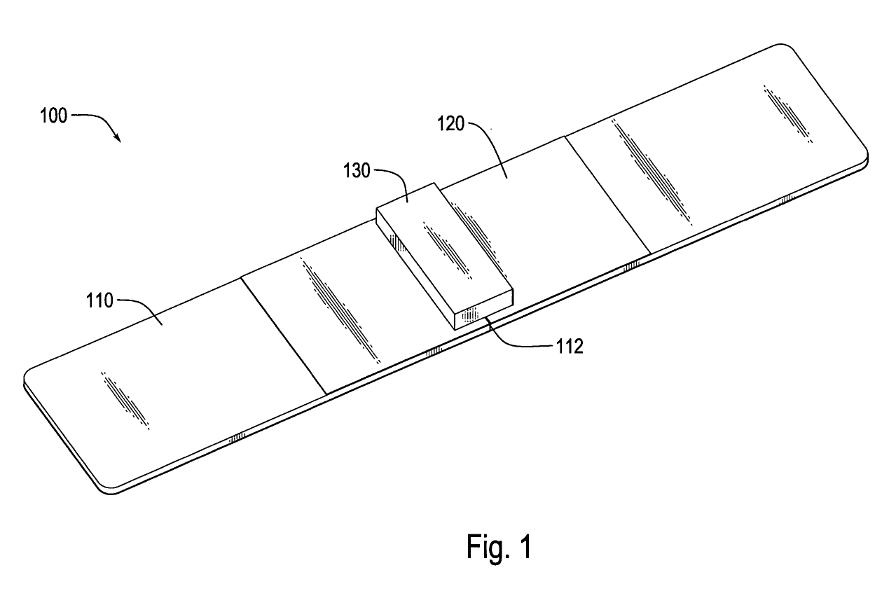

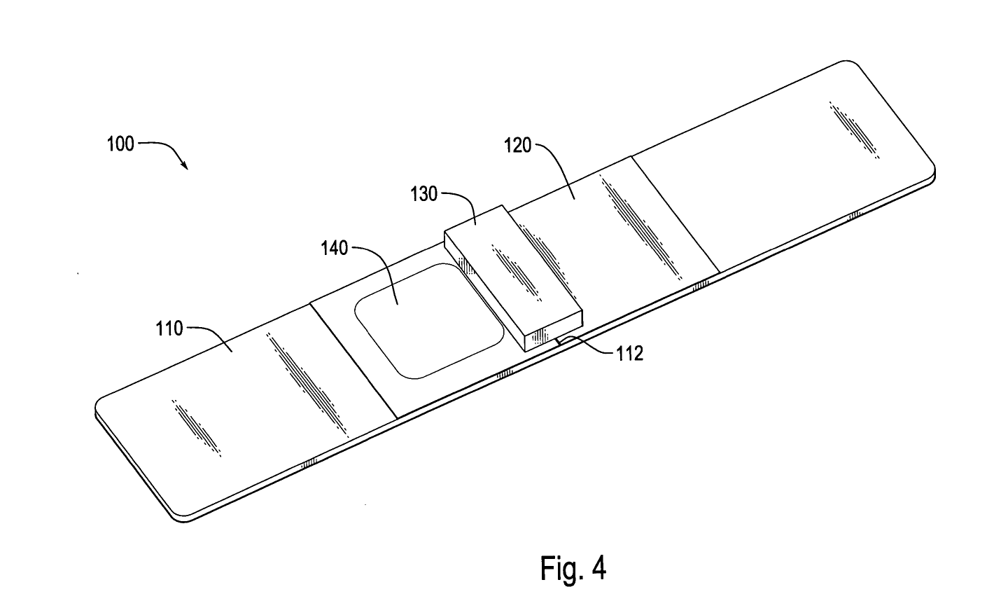

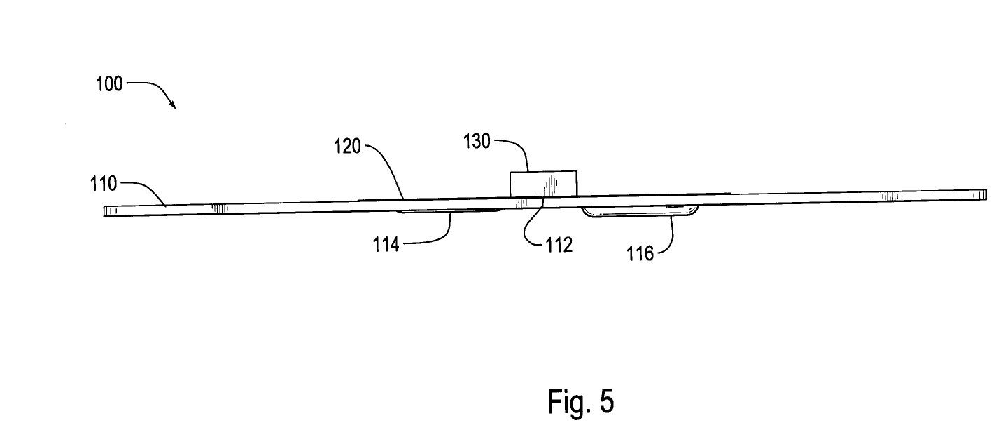

FIGS. 1-6 illustrate a first embodiment of this invention, although this invention is in no way limited to the specific design depicted therein. As shown in FIGS. 1-6, an applicator 100 has a semi-rigid base strip 110 on which a reservoir such as a sachet 120 is disposed. Further, an applicator tip such as a foam pad 130 is disposed over at least a portion of the sachet 120. The sachet 120 contains a single-use amount of an adhesive material 140 sealed therein.

“Semi-rigid” as used herein means that the base strip 110 is flexible enough to bend when force is exerted on it, for example, by hand. Absent sufficient pressure, the base strip retains its shape and supports the sachet 120 and the foam pad 130 thereon. Further, as described below, the base strip is rigid enough to provide a suitable handle for the applicator in use, and in the first exemplary embodiment, to apply pressure on the reservoir during use.

Thus, the base strip 110 may be made of any suitable material, such as plastic, aluminum, cardboard, or the like. Still, a weakened portion 112 may be provided in the base strip 110 to define an axis about which the base strip 110 may be bent. For example, as shown in the first embodiment, the weakened portion 112 is a cut in a surface of the base strip 110.

The applicator tip 130 may also be made of any suitable material and may be of any suitable configuration, for example, in accordance with the intended use of the applicator. Further, it is possible to incorporate one or more polymerization initiators or rate modifiers on or in the applicator tip, or elsewhere in the applicator, to initiate or modify the polymerization rate when polymerizable material is contained in the reservoir of the applicator.

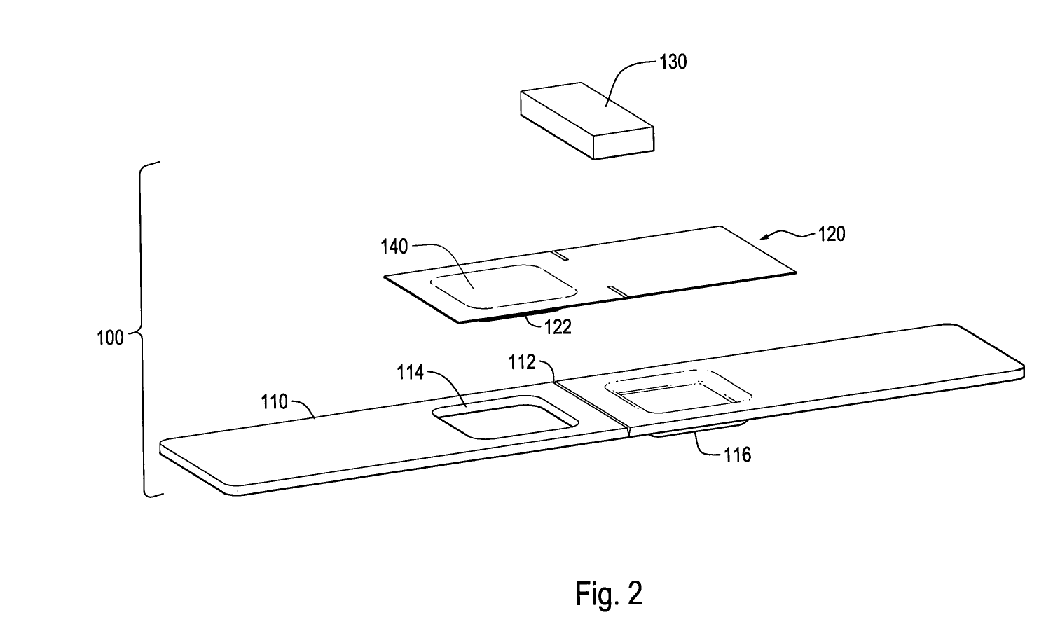

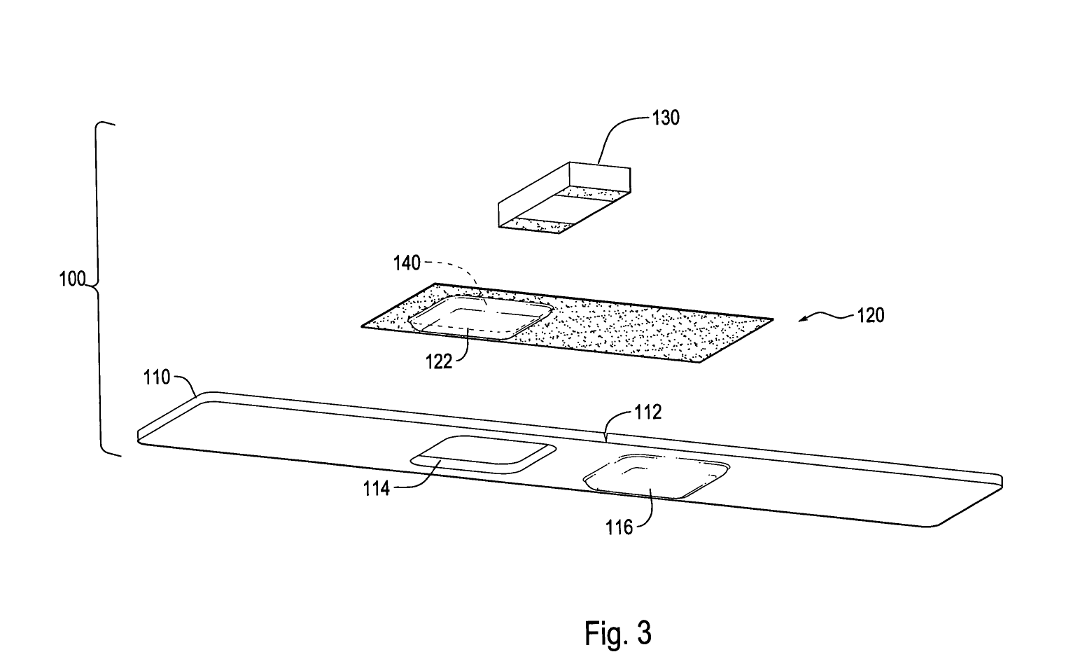

As shown in FIGS. 2 and 3, the base strip 110, the sachet 120 and the applicator tip 130 may be assembled to form the applicator 100. As illustrated by cross hatching, the sachet 120 may be secured to the base strip 110, for example, by a suitable adhesive, such as a pressure sensitive adhesive, hot melt adhesive, hot cure adhesive, or the like. Similarly, the applicator tip 130 may be secured to either the sachet 120 or the base strip 110. As illustrated, the applicator tip 130 may be secured only at the edges thereof. Thus, the central portion of the applicator tip 130 is not prevented or hindered from directly receiving the adhesive material 140 when released from the sachet 120. However, it will be readily apparent and understood that the sachet may be secured to the base strip in a variety of other manners, not limited to the cross-hatched adhesive shown in the Figures. Thus, for example, adhesive materials can be used in different configurations to adhere the sachet to the base strip, or the sachet can be adhered to the base strip in other different manners, such as pressure-fitting, snap-fitting between matching male and female portions, mechanical attachments such as clips, screws, or the like, and the like. The specific means of attachment is not particularly limited, although it preferably retains the sachet on the base strip in a manner to permit effective use of the applicator.

In various embodiments, the adherence of the sachet 120 to the base strip 110 is such that the sachet 120 will be ruptured under stress that is generated by stretching of the sachet 120 as the base strip 110 is folded during use, as described below. For example, at least portions of the sachet 120 on opposite sides of a portion of the sachet 120 that is to be ruptured are adhered to the base strip 110. As the base strip 110 is folded, the adhered portions of the sachet 120 then move with respective portions of the base strip 110 on opposite sides of the axis of folding so that the sachet 120 is stretched.

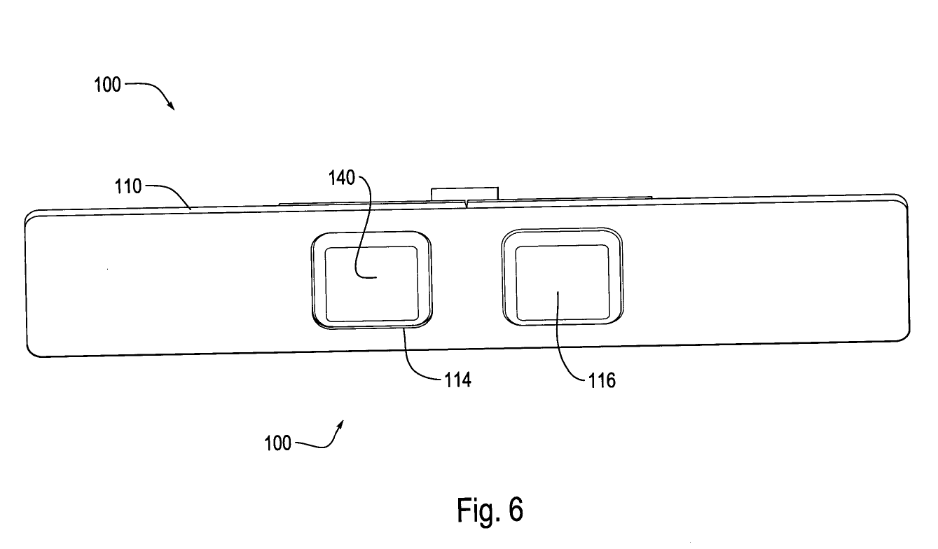

The base strip 110 has a hole 114 formed therethrough. As shown in FIGS. 3 and 6, the base strip 110 also has a corresponding boss or projection 116. The hole 114 and the projection 116 are located on opposite sides of the weakened portion 112 that defines an axis about which the base strip 110 is designed to fold. Thus, when the base strip 110 is folded, the projection 116 aligns with the hole 114 and may extend into the hole 114. Although the hole and boss are shown in the figures as generally square-shaped features, it will be apparent and understood that these features are not limited to this particular shape, and that varying shapes and designs can readily be used to accomplish the stated function. Further, while a single projection and a single hole are shown in the exemplary embodiment illustrated in the Figures, it will be apparent that multiple projections and/or holes may be provided.

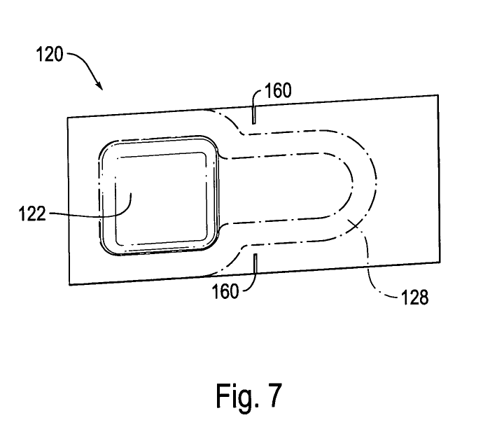

As shown in FIGS. 2, 3, 7 and 8, the sachet 120 has a formed portion 122 that, when the applicator 100 is assembled, fits into the hole 114 formed through the base strip 110, as shown in FIG. 6. As illustrated by the lack of crosshatching, the formed portion 122 of the sachet 120 is not secured to the base strip 110 so that the formed portion may easily deform during use of the applicator 100.

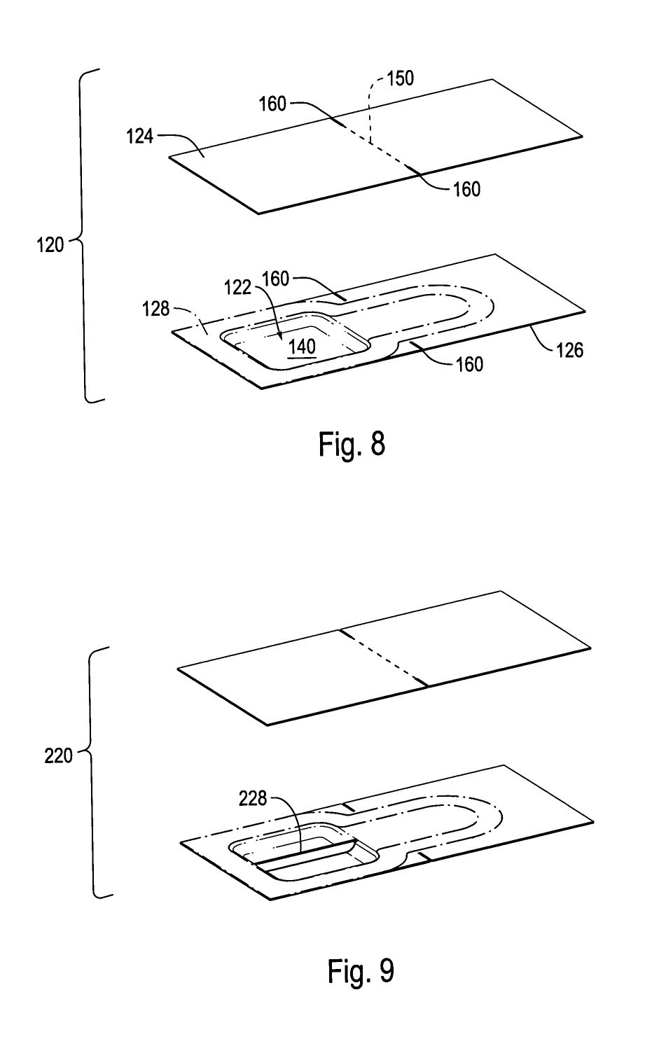

Further, as shown in FIG. 8, the sachet 120 may be made by sealing a first sheet of material 124 to a second sheet of material 126 along a peripheral path 128 that defines a cavity of the sachet 120 for containing the adhesive material 140. The seal may be achieved using any suitable known or hereafter developed technique for adhering, bonding or otherwise fixing the first and second sheet materials 124, 126 together. For example, the first and second sheet materials 124, 126 may be heat sealed or may be fixed together by an ultrasonic weld along the peripheral path 128. However, the sealing of the first and second sheet materials should be such that a complete environmental seal is formed, to seal the polymerizable material in the formed sachet and, as much as possible, prohibit premature contact of the polymerizable material with the outside environment. To the extent that a sealing means is used that may contact the contained polymerizable material, such as a separate adhesive material, it is preferred that the sealing means is compatible with the polymerizable material, such that it does not itself cause premature polymerization or otherwise adversely affect the contained polymerizable material.

The sachet 120 has a weakened portion that may be defined by a pre-scoring 150 of the first sheet material 124 and/or one or more slits 160 formed in an edge of the sachet 120. The weakened portion determines a portion that is to be ruptured during use.

Another exemplary embodiment of a sachet according to this invention is shown in FIG. 9. The sachet 220 is similar to the sachet described above, but has a partition 228 that separates the cavity of the sachet 220 into first and second compartments. In embodiments, the partition 228 may be formed as part of the sachet 220. Further, the partition 228 may be formed as a frangible barrier or the sachet may be formed to have multiple cavities.

As described below with respect to the sachet, the partition 228 is preferably made of a material that promotes the stability of the adhesive material, especially when the adhesive material is a polymerizable monomer composition. The first compartment may contain the adhesive material and the second compartment may contain an initiator or rate modifier to promote polymerization and/or cross-linking of the polymerizable adhesive material in use.

Additionally or alternatively, the second compartment may contain a medicament, an anesthetic and/or other material to be applied. In such a case, the applicator allows easy application of the adhesive material, and a second material as desired, that may be accomplished at the same time with one hand. The sachet 220 maintains the materials in a sealed condition prior to use and preferably leaves no excess adhesive material or other material since the applicator is designed for a single use. Examples of such medicaments include, but are not limited to antibiotics, antimicrobials, antiseptics, bacteriocins, bacteriostats, disinfectants, steroids, anesthetics, fungicides, anti-inflammatory agents, antibacterial agents, antiviral agents, antitumor agents, growth promoters, and mixtures thereof.

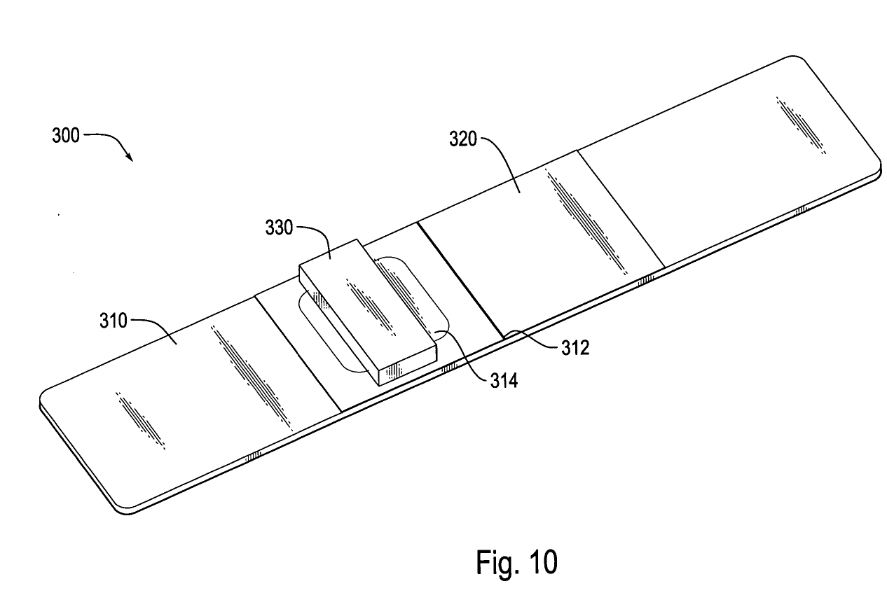

A second exemplary embodiment of this invention is shown in FIG. 10. As shown, an applicator 300 has a semi-rigid base strip 310 on which a reservoir such as a sachet 320 is disposed. Further, an applicator tip such as a foam pad 330 is disposed over at least a portion of the sachet 320. The sachet 320 contains a single-use amount of an adhesive material sealed therein.

The second embodiment of FIG. 10 is similar to the first embodiment of FIG. 1. In the second embodiment, the foam pad 330 is again located over the weakened portion of the sachet 320 that is to be ruptured. However, the weakened portion of the sachet 320 is offset or located away from the weakened portion 312 of the base strip 310. Thus, the weakened portion of the sachet 320 may be more closely aligned with the hole 314 of the base strip 310. Further, this provides a different configuration of the applicator 300 which may be useful for certain applications.

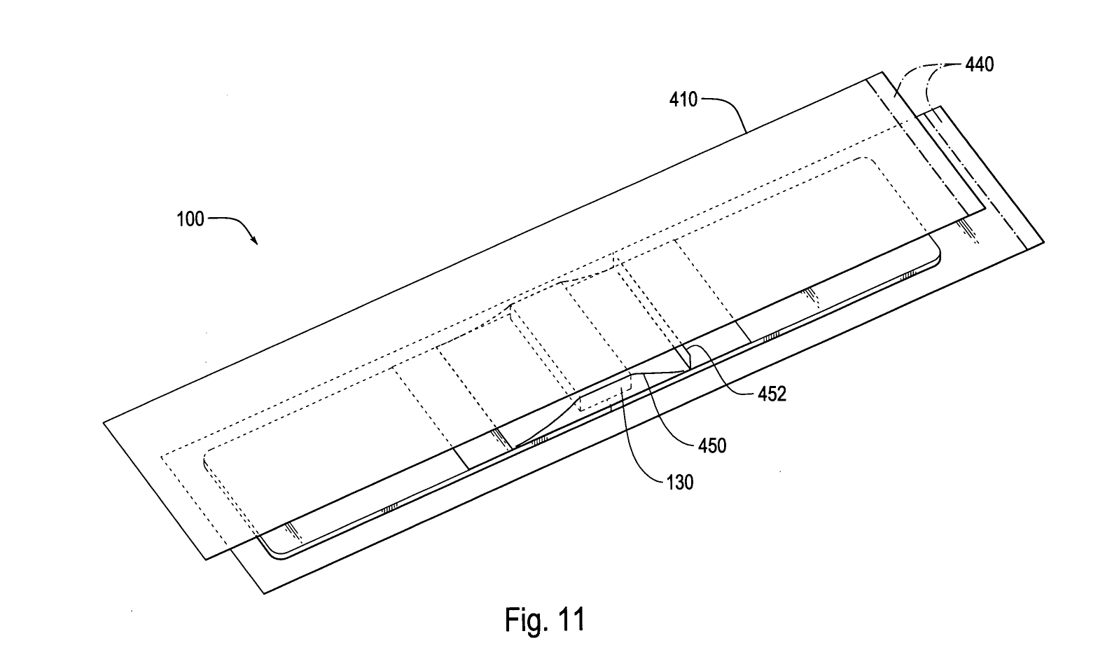

The applicators of this invention may include a packaging system. An exemplary embodiment of a packaging system 400 is shown in FIG. 11. The packaging system 400 comprises a pouch formed by a first sheet of material 410 and a second sheet of material 420. The sheets of material 410, 420 may be of any suitable material, such as, for example, polyester (e.g., Mylar®), metal foil or paper. Further, while two sheets of material are shown, a single sheet of material may be formed into the pouch as well.

The sheets of material 410, 420 may be attached to each other at a periphery 430 thereof by any suitable technique. For example, a releasable adhesive may be used. Further, part of a corner or part of an edge 440 of the sheets of material 410, 420 may be left unattached to facilitate separation of the sheets of material 410, 420 to remove the applicator 100 surrounded thereby.

Alternatively or additionally, the packaging system 400 may include a peel-off film 450 that covers at least the applicator tip 130 of the applicator 100. The peel-off film 450 may include a tab 452 to facilitate removal of the film 450 from use of the applicator 100. Similar techniques used to attach the sheets of material 410, 420 may be used to attach the peel-off film 450 to the applicator 100. In embodiments, the peel-off film 450 may be used to cover the adhesive after it has been applied to a desired surface.

The peel-off film 450 may seal the applicator tip 130 and may form a sterile barrier over the applicator tip 130. Further, in embodiments, the peel-off film 450 may be used without the sheets of material 410, 420, thus reducing waste and reducing manufacturing costs.

When the peel-off film 450 is used to cover the adhesive after it has been applied to a desired surface, the peel-off film 450 serves as a masking product over the adhesive. Additionally, the peel-off film 450 may include a design, decoration or other ornamental feature for aesthetic purposes. For example, the peel-off film 450 may be decorated with a cartoon character for aesthetic appeal to children of all ages.

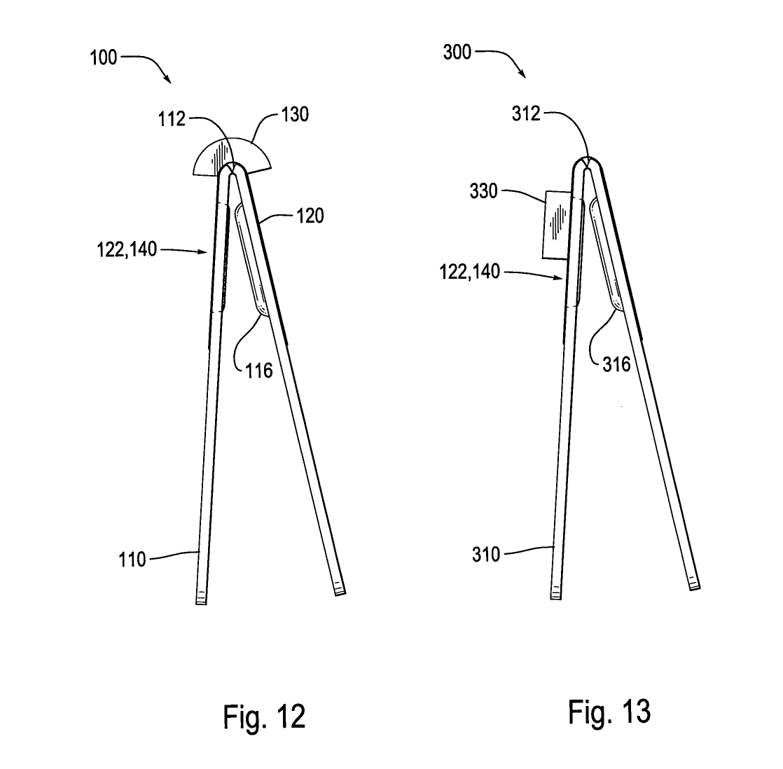

FIGS. 12 and 13 illustrate the applicators of FIGS. 1 and 10, respectively, when in use. After the applicator 100, 300 is removed from the packaging system, the base strip 110, 310 is folded about the weakened portion 112, 312 in a direction away from the applicator tip 130, 330. As the applicator 100, 300 is folded, the sachet 120, 320 is subjected to stress that ruptures the sachet 120, 320 at the weakened portion thereof. Further folding of the base strip 110, 310 applies additional pressure on the sachet 120, 320 to force the adhesive material 140 into/onto the applicator tip 130, 330.

In the exemplary embodiments described above, the projection 116, 316 is extended into the hole 114, 314 as the base strip 110, 310 is substantially completely folded. Thus, the projection 116, 316 applies pressure directly on the formed portion 122 of the sachet 120, 320 to expel the adhesive material 140. The folded base strip 110, 310 may be used as a handle to apply the adhesive 140 from the applicator tip 130, 330, facilitating accuracy of application and avoiding unwanted user contact with the adhesive 140.

The sachet or reservoir in which the adhesive material is held is preferably made of a material that tends to stabilize the adhesive, especially when the adhesive is a polymerizable monomer composition. Thus, it is possible and preferred in embodiments of the present invention that the adhesive composition does not include, or is substantially free of, one or more stabilizer components such as are known and used in the art as additives to the adhesive monomer composition. This may reduce, or even eliminate, the need for a polymerization initiator or accelerator.

For example, suitable materials include halogenated materials, preferably fluorinated materials. For example, where the reservoir is formed from a plastic or similar material, it is preferred that the reservoir, or at least a surface portion thereof that will be in contact with the adhesive monomer composition, is formed from a halogenated plastic material or is coated by a halogenated material. The ability of such halogenated polymers to provide barrier properties, and contribute to stability of adhesive compositions, is described in further detail in copending U.S. Application No. 09/430,289, filed Oct. 29, 1999, the entire disclosure of which is incorporated herein by reference. Likewise, in embodiments, the materials can include functionalized polymeric materials, such as are disclosed in the '289 application, where the functionalization provides the desired stabilizing effect to the adhesive composition. Of course, in embodiments, it may be desirable and/or necessary to add a separate stabilizer component to the adhesive composition, either to completely stabilize or fine-tune the stabilization characteristics of the adhesive composition.

Where polymer materials are used to form the reservoir, the reservoir may comprise a halogenated, preferably fluorinated, polymer on at least the internal surface of the reservoir, at least in the area in contact with the adhesive monomer composition. As used herein, a “halogenated polymer” can be any halogenated polymer that is known or becomes known in the art or can be any polymeric material that is suitable for fabrication of packaging that is subsequently or concurrently halogenated by at least one known halogenation method. However, the halogenation process must not render the polymeric material unusable as a reservoir material. As used herein, a “fluorinated polymer” is thus a halogenated polymer, wherein the halogen comprises, in whole or in part, fluorine.

The reservoir preferably comprises any suitable halogenated polymeric material, including, but not limited to, polyolefins, halogenated hydrocarbons (halocarbons), and engineered resins. The reservoir can comprise homopolymers, copolymers, higher order polymers, or mixtures thereof, and can comprise one species of polymeric material or mixtures of multiple species of polymeric material. As desired and/or necessary, the polymeric materials can be halogenated or otherwise functionalized either prior to manufacture of the reservoir, during manufacture of the reservoir, or subsequent to manufacture of the reservoir. Pre-halogenated (or pre-functionalized) materials are generally those that are already halogenated or functionalized, such as where halogenated or functionalized monomers are used to form the polymer reservoir. Concurrently halogenated or functionalized materials are those where although the precursor materials may not themselves be halogenated or functionalized, the halogenation or functionalization is introduced during the manufacturing process. For example, where the reservoir is made by molding, the halogenation or functionalization can be introduced by using a reactive halogen-containing gas. Likewise, post-halogenated or post-functionalized materials are those where the reservoir is first prepared, and then the formed polymeric material is subsequently halogenated or functionalized.

Reservoir materials of the present invention can, for example, comprise polyolefin polymers. Suitable polyolefins include, but are not limited to, polyethylene (PE), such as high-density polyethylene (HDPE), medium-density polyethylene (MDPE); low-density polyethylene (LDPE), cross-linked high-density polyethylene (XLPE), linear low-density polyethylene (LLDPE), ultra low-density polyethylene, and very low-density polyethylene; polycarbonate (PC); polypropylene (PP); polypropylene copolymer (PPCO); polyallomer (PA); polymethylpentene (PMP or TPX); polyketone (PK); polyethylene terephthalates (PET), including polyethylene terephthalate G copolymer (PETG) and oriented PET; polystyrene (PS); polyvinylchloride (PVC); naphthalate; polybutylene terephthalate; thermoplastic elastomer (TPE); mixtures thereof; and the like. Exemplary densities of the above polyethylenes are as follows: LDPE-0.910-0.925 g/cm³; medium-density polyethylene (MDPE)-0.926-0.940 g/cm³; HDPE-0.941-0.965 g/cm³. Other densities can be determined by the ordinary artisan by referencing, for example, ASTM D 1248 (1989).

The reservoir of this invention can comprise halogenated hydrocarbons (also referred to herein as halocarbons). For example, exemplary fluorinated hydrocarbons include, but are not limited to, Halar® ethylene-chlorotrifluoroethylene copolymer (ECTFE) (Allied Chemical Co., Morristown, N.J.); Tefzel® ethylene-tetrafluoroethylene (ETFE) (duPont, Wilmington, Del.); tetrafluoroethylene (TFE); polytetrafluoroethylene (PTFE); fluorinated ethylene propylene (FEP); polytetrafluoroethylene fluorinated ethylene propylene (PTFE-FEP); polyvinyl fluoride (PVF); polytetrafluoroethylene perfluoroalkoxy (PTFE-PFA); polyvinylidene fluoride (PVDF); mixtures thereof; and the like.

The reservoir of this invention can comprise engineered resins. Exemplary engineered resins include, but are not limited to, polyamide, such as nylon; polyphenylene oxides (PPO); polysulfone (PSF); mixtures thereof; and the like. In particular, relatively pure resins, such as Allethon 6017 and Marlex 5250 is highly desirable for stability of the polymerizable adhesive material.

In embodiments, the reservoir of this invention can comprise mixtures of the above polyolefins, halogenated hydrocarbons, and/or engineered resins.

However, the present invention is not limited to the reservoir or sachet being formed from such halogenated polymeric materials. Rather, the sachet can alternatively be formed from any suitable material that exhibits compatibility with the contained polymerizable material, at least for a desired shelf-life. Thus, for example, the sachet or reservoir can be formed of other known materials, such as aluminum, non-halogenated polymers, or the like.

Although the exemplary embodiments discussed above show the applicator tip as a foam pad, this is not limited to such embodiments. In particular, any suitable applicator tip can be used according to the present invention. Such suitable applicator tips include swabs, brushes, spatulas, droppers, syringes, and the like. Any suitable applicator tip can be used that allows for application of the adhesive composition to the desired site, and thus different applicator tips may be appropriate for different application methods.

The applicator tip can be composed of any of a variety of materials including polymerized materials such as plastics, foams, rubber, thermosets, films, or membranes. Additionally, the applicator tip may be composed of materials such as metal, glass, paper, ceramics, cardboard, and the like. The applicator tip material may be porous, absorbent, or adsorbent in nature to enhance and facilitate application of the adhesive composition. In general, the only limitation on the materials used to fabricate the tip is that the tip must be sufficiently compatible with the composition to be dispensed that undesirable effects on the composition do not prevail during contact of the composition with the tip. Various suitable designs for applicator tips that may be used according to this invention are disclosed in, for example, U.S. patent application Ser. No. 08/488,411, filed Jun. 7, 1995, Ser. No. 09/069,979, filed Apr. 30, 1998, Ser. No. 09/069,875, filed Apr. 30, 1998, and Ser. No. 09/385,030, filed Aug. 30, 1999, the entire disclosures of which are incorporated herein by reference.

In embodiments of this invention, it is preferred that all of the components that contact the polymerizable monomeric adhesive material include or are made from materials that are compatible with the monomer, as discussed above. Thus, the reservoir, at least in the area around the polymerizable monomeric adhesive material, and optionally the applicator tip and the applicator itself, preferably include such materials.

Furthermore, the applicator of this invention can be provided in any of various sizes, depending on the desired use of the product. Larger or smaller sizes can be used, where the sizes are tailored to the shape of the tip and/or the amount of adhesive material to be applied for a given application. Thus, for example, where the applicator is intended for applications requiring a large amount of adhesive material, a larger applicator can be used; whereas where the applicator is intended for applications requiring only a small amount of adhesive material, a smaller applicator can be used. Tailoring the size of the applicator to the amount of adhesive required can help prevent waste of adhesive material.

Further, multiple applicators can be provided where each of the applicators contains the same amount of adhesive composition, or where different amounts of adhesive composition are provided. Thus, a kit containing a plurality of applicators may be provided. Further, a kit can be provided having one or more applicators, such as of different sizes, shapes or constructions.

The applicators of this invention may be used to apply the polymerizable adhesive composition to a variety of substrates for the purposes of protecting, sealing, and bonding surfaces together. Suitable substrates include, but are not limited to, metals, plastics, rubbers, wood, ceramics, fabrics, cement, paper, living tissue and the like. For example, the polymerizable and/or cross-linkable material may be useful as tissue adhesives, sealants for preventing bleeding or for covering open wounds, systems for delivery of therapeutic or other bioactive agents, and other biomedical applications. They find uses in, for example, closing surgically incised or traumatically lacerated tissues; setting fractured bone structures; retarding blood flow from wounds; aiding repair and regrowth of living tissues; dressing burns; dressing skin or other superficial or surface wounds (such as abrasions, chaffed or raw skin, and/or stomatitis); and protecting tissues prone to damage (e.g., as artificial calluses).

The polymerizable adhesive composition, in embodiments, is preferably a monomeric (including prepolymeric) adhesive composition. In embodiments, the monomer is a 1,1-disubstituted ethylene monomer, e.g., an α-cyanoacrylate. Preferred monomer compositions of the present invention, and polymers formed therefrom, are useful as tissue adhesives, sealants for preventing bleeding or for covering open wounds, and in other absorbable and non-absorbable biomedical applications. They find uses in, for example, apposing surgically incised or traumatically lacerated tissues; retarding blood flow from wounds; drug delivery; dressing burns; dressing skin or other superficial or surface wounds (such as abrasions, chaffed or raw skin, and/or stomatitis); hernia repair; meniscus repair; and aiding repair and regrowth of living tissue. Other preferred monomer compositions of the present invention, and polymers formed therefrom, are useful in industrial and home applications, for example in bonding rubbers, plastics, wood, composites, fabrics, and other natural and synthetic materials.

The monomer (including prepolymeric) adhesive composition may include one or more polymerizable monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687 and 5,928,611 to Leung et al., U.S. patent application Ser. No. 09/430,177, filed on Oct. 29, 1999, and U.S. patent application Ser. No. 09/471,392 filed Dec. 23, 1999, which are hereby incorporated in their entirety by reference herein. Other suitable monomers, and mixtures thereof, are disclosed in U.S. Patent Applications No. 09/630,437, filed Aug. 2, 2000, and Ser. No. 09/919,877, filed Aug. 2, 2001, the entire disclosures of which are incorporated herein by reference. Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 2 to about 12 or more preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

As desired, the applicators according to this invention can include any of a wide variety of additional materials, either mixed into the polymerizable composition, or in a separate compartment from the polymerizable composition. Examples of suitable additional materials include, but are not limited to, plasticizing agents, thixotropic agents, thickeners, natural or synthetic rubbers, stabilizers, pH modifiers, bioactive agents, cross-linking agents, chain transfer agents, fibrous reinforcements, colorants, preservatives, formaldehyde reducing or scavenging agents, flavorants, perfumes, mixtures thereof, and the like.

The composition may optionally also include at least one other plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to tributyl citrate, acetyl tri-n-butyl citrate (ATBC), polymethylrnethacrylate, polydimethylsiloxane, hexadimethylsilazane, isopropyl myristate, isopropyl palmitate, and others as listed in U.S. patent application Ser. No. 09/471,392 filed Dec. 23, 1999, the disclosure of which is incorporated in its entirety by reference herein.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. patent application Ser. No. 09/374,207 filed Aug. 12, 1999, the disclosures of which are hereby incorporated in their entireties by reference herein.

The composition may optionally also include thickeners. Suitable thickeners may include poly (2-ethylhexy methacrylate), poly(2-ethylhexyl acrylate) and others as listed in U.S. patent application Ser. No. 09/471,392 filed Dec. 23, 1999, and Ser. No. 09/374,207, filed Aug. 12, 1999, the disclosures of which are incorporated by reference herein in their entirety.

The composition may optionally also include one or more stabilizers, preferably both at least one anionic vapor phase stabilizer and at least one anionic liquid phase stabilizer. The composition may optionally also include, in addition to or in place of the anionic stabilizers, at least one free radical stabilizer. These stabilizing agents may inhibit premature polymerization. Suitable anionic and free radical stabilizers may include those listed in U.S. patent application Ser. No. 09/471,392 filed on Dec. 23, 1999, and Ser. No. 09/099,457, filed Jun. 18, 1998, the disclosures of which are incorporated by reference herein in their entirety.

However, as described above, a particular advantage of this invention, such as in embodiments where stabilizing materials are used, is that separate stabilizers can be omitted from the composition. Thus, in embodiments, the polymerizable composition preferably does not include any, or at least substantially none, additional stabilizer.

The compositions may also include pH modifiers to control the rate of degradation of the resulting polymer, as disclosed in U.S. patent application Ser. No. 08/714,288, filed Sep. 18, 1996, the entire disclosure of which is hereby incorporated by reference herein in its entirety.

Compositions of this invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites, etc. Additional examples of formaldehyde scavenger compounds useful in this invention and methods for their implementation can be found in U.S. Pat. Nos. 5,328,687, 5,514,371, 5,514,372, 5,575,997, 5,582,834 and 5,624,669, all to Leung et al., which are hereby incorporated herein by reference in their entireties.

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain fibrous reinforcement and colorants such as dyes, pigments, and pigment dyes. Examples of suitable fibrous reinforcement include PGA microfibrils, collagen microfibrils, and others as described in U.S. patent application Ser. No. 09/471,392 filed on Dec. 23, 1999, the disclosure of which is incorporated by reference herein in its entirety.

The polymerizable compositions useful in this invention may also further contain one or more preservatives, for prolonging the storage life of the composition. Suitable preservatives, and methods for selecting them and incorporating them into adhesive compositions, are disclosed in U.S. patent application Ser. No. 09/430,180, the entire disclosure of which is incorporated herein by reference.

In embodiments of this invention, the composition and/or its applicator or dispenser may contain additional materials such as a polymerization initiator, accelerator, rate-modifier, and/or cross-linking agent for initiating polymerization and/or cross-linking of the polymerizable monomer material. Such initiators, accelerators, rate-modifiers, and/or cross-linking agents can be included in the applicator tip, in the polymerizable composition, and/or elsewhere, as appropriate.

In embodiments of this invention, it is possible to incorporate into the applicator tip additional components, such as polymerization initiators and/or accelerators, anesthetic, medicament or the like, or even any of the various additives described above with respect to the polymerizable composition. This is advantageous, for example, where additional initiator or accelerator may be necessary to provide the desired cure rate of the adhesive once it is applied or where additional treatment is desired. Furthermore, this is advantageous in embodiments where additional stabilizers or polymerization inhibitors must be added to the adhesive composition in the assembly, so as to overcome the “cure speed loss” that often occurs when such stabilizing agents are added.

In embodiments, the initiator or accelerator material is an initiator and/or a rate modifier for polymerization and/or cross-linking of a polymerizable monomer. As used herein, a polymerization initiator is any material that causes a monomer composition applied to a substantially dry tissue (i.e., substantially in the absence of plasma or like tissue fluids) to polymerize in less than 300 seconds at ambient temperature, for example, at approximately 21-25° C. Preferably, the initiator causes the monomer composition to polymerize in less than 150 seconds at ambient temperature, more preferably within 60, 90 or 130 seconds. As used herein, a polymerization rate modifier is any material that changes the rate at which a polymerizable monomer would polymerize in the absence of that material. Preferably, the rate modifier accelerates the rate of the polymerization reaction, although for particularly fast-acting monomers it may decelerate that rate.

The material may be applied to the applicator tip, for example, by spraying, dipping, injecting, or brushing the applicator tip with a liquid medium containing the polymerization initiator or accelerator. It is preferably applied to the tip by dipping or injecting. For example, it may be applied to the tip by pumping of the liquid medium, for example, through a syringe, onto the tip. Methods of applying the polymerization initiator or accelerator to an applicator tip are described in more detail in U.S. Pat. No. 5,928,611 to Leung and U.S. patent application Ser. No. 09/069,979, filed Apr. 30, 1998, Ser. No. 08/920,876, filed Aug. 29, 1997, and Ser. No. 09/430,177, filed Oct. 29, 1999, the entire disclosures of which is incorporated herein by reference.

As described above, an advantage of this invention is that the applicators can be provided in various single-use sizes, based on the desired or intended uses of the adhesive compositions. In such embodiments, the applicator tip size and/or the amount of polymerizable adhesive composition can be selected from various alternatives. This concept further applies to the amount of polymerization initiator or accelerator that can be added. For example, the amount can be adjusted based on the desired or intended uses of the adhesive compositions. Thus, for example, where an application would require only a small amount of adhesive composition, a correspondingly small amount of initiator or accelerator can be applied to an appropriately sized applicator tip or included in an appropriately sized separate cavity in the reservoir; likewise, where an application would require a larger amount of adhesive composition, a correspondingly larger amount of initiator or accelerator can be applied to an appropriately larger applicator tip or included in an appropriately sized separate cavity in the reservoir.

Particular initiators and accelerators for particular monomers may be readily selected by one of skill in the art without undue experimentation. Control of the molecular weight distribution of the applied adhesive can be enhanced by selection of the concentration and functionality of the initiator or accelerator vis-a-vis the selected monomer. Suitable polymerization initiators and accelerators for cyanoacrylate compositions include, but are not limited to, detergent compositions; surfactants, including nonionic surfactants such as polysorbate 20 (e.g., Tween 20™; ICI Americas), polysorbate 80 (e.g., Tween 80™; ICI Americas), and poloxamers; cationic surfactants such as tetrabutylammonium bromide; anionic surfactants, including quaternary ammonium halides such as benzalkonium chloride or its pure components, and benzethonium chloride; stannous octoate (tin (II) 2-ethylhexanoate), and sodium tetradecyl sulfate; and amphoteric or zwitterionic surfactants such as dodecyldimethyl(3-sulfopropyl) ammonium hydroxide, inner salt; amines, imines, and amides, such as imidazole, tryptamine, urea, arginine and povidine; phosphines, phosphites and phosphonium salts, such as triphenylphosphine and triethyl phosphite; alcohols such as ethylene glycol; methyl gallate; inorganic bases and salts, such as sodium bisulfite, magnesium hydroxide, calcium sulfate and sodium silicate; sulfur compounds such as thiourea and polysulfides; polymeric cyclic ethers such as monensin, nonactin, crown ethers, calixarenes and polymeric epoxides; cyclic and acyclic carbonates, such as diethyl carbonate; phase transfer catalysts such as Aliquat™ 336 (General Mills, Inc., Minneapolis, Minn.); organometallics; manganese acetylacetonate; radical initiators and radicals, such as di-t-butyl peroxide and azobisisobutyronitrile; and bioactive compounds or agents.

In preferred embodiments, the initiator may be a bioactive material, including quaternary ammonium halides such as alkylbenzyldimethylammonium chloride (benzalkonium chloride; BAC) its pure components, or mixtures thereof, especially those with an alkyl containing 6-18 carbon atoms; benzethonium chloride; and salts of sulfadiazine. Cobalt napthenate can be used as an accelerator for peroxide. Other suitable bioactive materials are disclosed in U.S. Pat. No. 5,928,611 to Leung and U.S. patent application Ser. No. 08/920,876, filed Aug. 29, 1997, Ser. No. 09/430,176 filed Oct. 29, 1999, and Ser. No. 09/430,177, filed Oct. 29, 1999, the entire disclosures of which is incorporated herein by reference.

The polymerizable adhesive compositions according to this invention can also comprise a medicament. Inclusion of a medicament is often desirable in compositions intended for medical applications. The medicament can either be added to the monomer-containing adhesive composition prior to packaging, or, alternatively, to the applicator tip or a separate compartment. Thus, the medicament may be applied to a tissue prior to or simultaneously with application of the monomer-containing adhesive composition. In addition to serving its medicinal function, the medicament may be selected so that it functions in conjunction with the co-packaged polymerizable monomer composition to initiate polymerization of the monomer or modify (e.g., accelerate) the rate of polymerization for the monomer to form a polymeric adhesive. The proper combination of medicament and polymerizable monomer can be determined easily by one of skill in the art. The medicament is supplied in an amount that will be pharmaceutically effective when applied topically (i.e., directly to tissue).

Examples of such medicaments include, but are not limited to antibiotics, antimicrobials, antiseptics, bacteriocins, bacteriostats, disinfectants, steroids, anesthetics, fungicides, anti-inflammatory agents, antibacterial agents, antiviral agents, antitumor agents, growth promoters, and mixtures thereof.

Exemplary medicaments include, but are not limited to, quaternary ammonium halides such as benzalkonium chloride and benzethonium chloride; chlorhexidine sulfate; gentamicin sulfate; hydrogen peroxide; quinolone thioureas; silver salts, including, but not limited to, silver acetate, silver benzoate, silver carbonate, silver chloride, silver citrate, silver iodide, silver nitrate, and silver sulfate; sodium hypochlorite; salts of sulfadiazine, including, but not limited to silver, sodium, and zinc salts; and mixtures thereof.

Preferable medicaments are those that are anions or help in radical generation or that are ion pairs or are themselves radicals.

In embodiments, the medicament is preferably a quaternary ammonium halide such as alkylbenzyldimethylammonium chloride (benzalkonium chloride; BAC) with an alkyl containing 6-18 carbon atoms, its pure components, or mixtures thereof, or benzethonium chloride; or a salt of sulfadiazine, such as a silver, sodium, or zinc salt.

The medicament can have a pharmaceutical effect only at the site of application (i.e., limited to the tissue on/in which it is applied), or it can have a systemic effect (by systemic, it is not only meant that the medicament has an effect throughout the patient's body, but also at a specific site other than the site of application). In embodiments where the medicament is applied in an amount sufficient to show a systemic pharmaceutical activity, it can be absorbed, transported, or otherwise distributed to the site or sites within the patient where the pharmaceutical activity is desired, e.g., through the cardiovascular or lymph systems. The medicament may be in the form of a solid, such as a powder or a solid film, or in the form of a liquid, such as a watery, viscous, or paste-like material. The medicament may also be compounded with a variety of additives, such as surfactants or emulsifiers, and vehicles.

The polymerizable and/or cross-linkable material may be neat (no additional compounds added) or in a solvent, emulsion or suspension. Suitable solvents according to the present invention include alcohol, ether alcohol, hydrocarbons, halogenated hydrocarbons, ethers, acetals, ketones, esters, acids, sulfur- or nitrogen-containing organic compounds, mixtures thereof and the like. Other suitable solvents are disclosed in U.S. Pat. No. 5,130,369 to Hughes et al. and U.S. Pat. No. 5,216,096 to Hattori et al., the entire disclosures of which are incorporated herein by reference. These solvents may be used either independently or in combination of two or more. They may also be used in conjunction with water to the extent that the polymerizable and/or cross-linkable material is dissolved or suspended in such a mixture. The total amount of solvent that may be incorporated into the polymerizable and/or cross-linkable material may be 0 to 99, preferably 1 to 50, and more preferably 3 to 25 percent by weight. Selection of the amount will, of course, depend on the desired monomer and process conditions, and amounts outside these ranges may be acceptable.

In embodiments, the monomer composition, the applicator and/or its packaging are preferably sterilized. Sterilization of the monomer composition, the applicator and/or its packaging can be accomplished by techniques known to one of ordinary skill in the art, and is preferably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. A preferred method is electron beam irradiation, as described in U.S. patent application Ser. No. 09/025,472, filed on Feb. 18, 1998, the entire disclosure of which is incorporated herein by reference. The composition must show low levels of toxicity to living tissue during its useful life. In preferred embodiments of the present invention, the composition is sterilized to provide a Sterility Assurance Level (SAL) of at least 10⁻³. In embodiments, the Sterility Assurance Level may be at least 10⁻⁴, or may be at least 10⁻⁵, or may be at least 10⁻⁶.

The polymerizable adhesive composition according to the invention can be manufactured and sterilized in very small quantities. Typically, sterilized α-cyanoacrylate compositions are sterilized in large volumes (e.g., 1-5 milliliters). When intended for medical applications, this large volume is undesirable because much of the composition is discarded after the first use out of fear of contamination of the composition. Thus, providing sterile α-cyanoacrylate compositions in smaller volumes is desirable. Thus, the sterilized compositions of embodiments of the invention provide an improvement over the sterile compositions currently available.

Preferably, a polymerizable adhesive composition according to this invention is packaged such that a total volume of no more than 1 mL of the adhesive composition is present per package (i.e., container). In embodiments, the total volume of the adhesive may be in the range of 0.01-5.0 mL. More preferably, the total volume of the adhesive may be in the range of 0.05-1.0 mL. Still more preferably, the total volume of the adhesive may be in the range of 0.07-0.12 mL. As noted above, such compositions of the invention can be sterilized by appropriate means, including, but not limited to, dry heat sterilization, gamma irradiation, microwave irradiation, and electron beam irradiation.

In embodiments where the compositions are to be used for medical applications, the sterilized composition must show low levels of toxicity to living tissue during its useable life. For example, sterilized compositions according to embodiments of the present invention show an increase in viscosity of no more than 300% as a result of sterilization. Viscosity levels can be determined by known techniques. For example, viscosity can be determined at room temperature (approximately 21-25° C.) using a Brookfield Cone-Plate Viscometer with spindle size CP-40. The instrument is standardized using a Viscosity Reference Standard in the same range as the sample to be tested. Each sample is measured three times, and an average value determined and recorded.

To be considered sterile, the polymerizable adhesive composition should show no bacterial growth after inoculation onto Soybean Casein Digest media, and incubation for 14 days at 32-35° C. Standard procedures and materials, such as those disclosed in USP XXIII<1211>, “Sterilization and Sterility Assurance of Compendial Articles” should be followed.

Preferably, the polymerizable adhesive composition has, immediately after sterilization, a viscosity level no more than 15-20% higher than the level prior to sterilization. However, the acceptable viscosity can be as high as 200% higher than the level prior to sterilization. More preferably, the sterilized composition has a viscosity that is no more than 50% higher than the viscosity of the composition before sterilization. Most preferably, the composition has a viscosity that is essentially unchanged from the level prior to sterilization (i.e., less than 20% higher). The acceptable viscosity after sterilization will need to be below 200% higher than the initial value in order for the monomeric adhesive composition to be of high utility in the application for which it is intended. In general, the increase in viscosity during sterilization can be viewed as “premature” aging of the monomer-containing composition, which reduces its useful shelf life, particularly when it is not stored at reduced temperature. In addition, the change in the viscosity is also an indication of a change in the reactivity of the monomeric composition, which normally is not desired.

In preferred embodiments, there is substantially no initiation of polymerization of monomeric liquid adhesive compositions that affects the utility of the monomer or monomers caused by the sterilization process. The sterilized liquid adhesive compositions have a good shelf life and excellent stability.

It should be understood that the individual features of the various exemplary embodiments may be included or excluded as desired for a given application. As such, all possible combinations of the described features are considered to be encompassed by the present invention.

Thus, while the present invention has been described in terms of exemplary embodiments, it is to be understood that the present invention is not to be limited to the particular configuration of these embodiments. One skilled in the art will recognize that various modifications and/or alterations of these embodiments may be made while remaining within the scope of the present invention.

1. An applicator for applying an adhesive material, comprising:
a semi-rigid base strip having a longitudinal direction, a hole formed through the base strip and a projection corresponding to the hole formed on one side of the base strip; and
a reservoir containing an adhesive material, the reservoir being disposed over the hole on a side of the base strip opposite the one side on which the projection is formed;
wherein the base strip is arranged to be folded about an axis substantially perpendicular to the longitudinal direction such that a portion of the reservoir is ruptured and the projection extends into the hole to apply pressure on the reservoir and expel the adhesive material from the ruptured reservoir.

2. The applicator according to claim 1, further comprising a medicament contained in the reservoir.

3. The applicator according to claim 1, wherein the adhesive material comprises a polymerizable monomer.

4. The applicator according to claim 1, wherein the adhesive material comprises a polymerizable 1,1-disubstituted ethylene monomer formulation.

5. The applicator according to claim 1, wherein the adhesive material comprises a cyanoacrylate formulation.

6. The applicator according to claim 3, wherein the reservoir is formed from a material that is compatible with the polymerizable monomer.

7. The applicator according to claim 6, wherein the reservoir is formed of aluminum.

8. The applicator according to claim 1, further comprising an applicator tip formed over at least the portion of the reservoir that is ruptured.

9. The applicator according to claim 8, wherein the applicator tip is at least one of porous, absorbent and adsorbent in nature.

10. The applicator according to claim 8, wherein the applicator tip comprises a material selected from the group consisting of metal, glass, paper, ceramics and cardboard.

11. The applicator according to claim 8, wherein the applicator tip comprises a plastic material.

12. The applicator according to claim 8, wherein the applicator tip comprises one of a foam pad, a rolling ball, a brush, a mesh, a spatula, a sponge and a swab.

13. The applicator according to claim 8, wherein at least a central portion of the applicator tip is not secured to the reservoir and is not secured to the base strip.

14. The applicator according to claim 8, further comprising a polymerization initiator or accelerator disposed in or on the applicator tip.

15. The applicator according to claim 14, wherein the applicator tip is at least one of porous, absorbent and adsorbent in nature, and the polymerization initiator or accelerator is absorbed or adsorbed into the applicator tip.

16. The applicator according to claim 8, further comprising a medicament disposed in or on the applicator tip.

17. The applicator according to claim 16, wherein the applicator tip is at least one of porous, absorbent and adsorbent in nature, and the medicament is absorbed or adsorbed into the applicator tip.

18. The applicator according to claim 8, wherein the applicator tip is sterilized.

19. The applicator according to claim 1, wherein the adhesive material is sterilized.

20. The applicator according to claim 1, wherein the portion of the reservoir that is ruptured is located substantially at the axis about which the base strip is arranged to be folded.

21. The applicator according to claim 1, wherein the portion of the reservoir that is ruptured is located away from the axis about which the base strip is arranged to be folded.

22. The applicator according to claim 1, wherein the portion of the reservoir that is ruptured is arranged to be ruptured by folding the base strip about the axis.

23. The applicator according to claim 22, wherein at least portions of the reservoir are adhered to the base strip so that folding the base strip about the axis applies stress on at least the portion of the reservoir that is ruptured.

24. The applicator according to claim 1, wherein the portion of the reservoir that is ruptured is arranged to be ruptured by pressure applied on the reservoir by the projection.

25. The applicator according to claim 1, wherein the projection is formed integral with the base strip.

26. The applicator according to claim 1, wherein the base strip has a weakened portion that defines the axis.

27. The applicator according to claim 26, wherein the weakened portion is a cut in a surface of the base strip.

28. The applicator according to claim 1, wherein the reservoir is a sachet.

29. The applicator according to claim 28, wherein the sachet has an ultrasonic weld seal.

30. The applicator according to claim 28, wherein the sachet has a weakened portion that defines the portion of the reservoir that is ruptured.

31. The applicator according to claim 30, wherein the weakened portion comprises a pre-scoring.

32. The applicator according to claim 30, wherein the weakened portion comprises a slit formed in at least one edge of the sachet.

33. The applicator according to claim 28, wherein the sachet has a formed portion that fits at least partially into the hole of the base strip.

34. The applicator according to claim 33, wherein the formed portion of the sachet is not secured to the base strip.

35. The applicator according to claim 1, wherein the reservoir is partitioned into a first compartment and a second compartment, the adhesive material being contained in the first compartment and a second material being contained in the second compartment.

36. The applicator according to claim 35, wherein the reservoir is partitioned by a frangible barrier.

37. The applicator according to claim 35, wherein the second material comprises a medicament.

38. The applicator according to claim 35, wherein the adhesive material comprises a polmerizable monomer and the second material comprises a polymerization initiator or accelerator.

39. A kit comprising a plurality of associated applicators of claim 1.

40. The kit of claim 39, wherein a first of the applicators comprises a greater amount of adhesive material than a second of the applicators.

41. The kit of claim 39, wherein a first of the applicators comprises a greater amount of polymerization initiator or accelerator than a second of the applicators.

42. The kit of claim 39, wherein a first of the applicators comprises a larger sized applicator tip than a second of the applicators.

43. An applicator for applying an adhesive material, comprising:
a semi-rigid base strip having a longitudinal direction; and
a sachet containing an adhesive material, the sachet being secured to one side of the base strip;
wherein the base strip is arranged to be folded about an axis substantially perpendicular to the longitudinal direction such that a portion of the sachet is ruptured and pressure is applied on the sachet to expel the adhesive material from the ruptured sachet.

44. A kit comprising a plurality of associated applicators of claim 43.

45. A method of applying an adhesive material, comprising:
folding the base strip of the applicator of claim 1;
rupturing the reservoir by the folding;
applying pressure on the reservoir to expel the adhesive material from the reservoir by extending the projection into the hole; and
directly applying the expelled adhesive material to a substrate to be bonded.

46. The method of claim 45, wherein the substrate to be bonded comprises tissue.

47. The method of claim 46, wherein the tissue has been burned.

48. The method of claim 46, wherein the tissue has been lacerated.

49. A method of applying an adhesive material, comprising:
folding the base strip of the applicator of claim 1;
rupturing the sachet by the folding;
applying pressure on the sachet to expel the adhesive material from the reservoir by the folding; and
directly applying the expelled adhesive material to a substrate to be bonded.

50. The method of claim 49, wherein the substrate to be bonded comprises tissue.

51. The method of claim 49, wherein the tissue has been burned.

52. The method of claim 49, wherein the tissue has been lacerated.

53. The applicator of claim 8, further comprising a peel-off film covering at least the applicator tip.

54. The applicator of claim 53, wherein the peel-off film is adhered to at least one of the reservoir and the base strip at a periphery of the peel-off film.

55. The applicator of claim 53, wherein the peel-off film has a tab outside of the periphery that is not adhered.

56. The applicator of claim 53, wherein the peel-off film seals the applicator tip.

57. The applicator of claim 56, wherein the peel-off film forms a bacterial barrier.

58. The applicator of claim 53, further comprising an outer packaging that surrounds the base strip, the reservoir and the applicator tip.

59. The applicator of claim 58, wherein the outer packaging seals the base strip, the reservoir and the applicator tip.

60. The applicator of claim 59, wherein the outer packaging forms a bacterial barrier.

61. The applicator of claim 58, wherein the outer packaging comprises:
a first sheet of material; and
a second sheet of material adhered to the first sheet of material at least along a periphery thereof.

62. The applicator of claim 61, wherein the second sheet of material is not adhered to the first sheet of material at an edge outside of the periphery.

63. A method of applying an adhesive material, comprising:
peeling off the peel-off film covering the applicator tip of the applicator of claim 53;
folding the base strip;
rupturing the reservoir by the folding;
applying pressure on the reservoir to expel the adhesive material from the reservoir by extending the projection into the hole; and
directly applying the expelled adhesive material to a substrate to be bonded.

64. The method of claim 63, further comprising removing an outer packaging that surrounds the base strip, the reservoir and the applicator tip prior to the peeling, rupturing and applying steps.

65. The method of claim 63, wherein peeling off the peel-off film is after rupturing the reservoir.
